(19) United States
(12) Patent Application Publication
(10) Pub. No.: US 2022/0031200 A1
(43) Pub. Date: Feb. 3, 2022

(54) Component Concentration Measuring Device

(71) Applicant: Nippon Telegraph and Telephone Corporation, Tokyo (JP)

(72) Inventors: Daichi Matsunaga, Tokyo (JP); Yujiro Tanaka, Tokyo (JP); Michiko Seyama, Tokyo (JP); Masahito Nakamura, Tokyo (JP)

(21) Appl. No.: 17/417,595

(22) PCT Filed: Dec. 11, 2019
(86) PCT No.: PCT/JP2019/048422

§ 371 Date: Jun. 23, 2021

(30) Foreign Application Priority Data
Dec. 25, 2018 (JP) 2018-240803

(51) Int. Cl. A61B 5/1455; G01N 29/02; G01N 29/24; A61B 5/11; G01N 33/49

(52) U.S. Cl. CPC A61B 5/1455; G01N 29/02; G01N 2291/02466; A61B 5/1126; G01N 33/49; G01N 29/2418

(57) ABSTRACT

A measurement unit is attached to a measurement subject and measures acceleration in time series. A body movement calculation unit obtains the magnitude of body movement indicating the magnitude of a motion of the measurement subject based on acceleration measured by the measurement unit. A singular point extraction unit extracts, as a singular point, a time point at which the magnitude of body movement obtained by the body movement calculation unit exceeds a threshold value. A matching unit rectifies a change in a photoacoustic signal between before and after the singular point extracted by the singular point extraction unit.

### CROSS-REFERENCE TO RELATED APPLICATIONS

This patent application is a national phase filing under section 371 of PCT/JP2019/048422, filed Dec. 11, 2019, which claims the priority of Japanese patent application no. 2018-240803, filed Dec. 25, 2018, each of which is incorporated herein by reference in its entirety.

### TECHNICAL FIELD

The present invention relates to a component concentration measuring device, and more specifically relates to a component concentration measuring device for non-invasively measuring the concentration of a component such as glucose in blood.

### BACKGROUND

Knowing (measuring) the blood glucose level is very important when determining an insulin dosage for a person with diabetes, preventing diabetes, and so on. The blood glucose level is the concentration of glucose in blood, and photoacoustics is a well-known method for measuring the concentration of this type of component (see PTL 1).

When a living body is irradiated with a certain amount of light (electromagnetic waves), the emitted light is absorbed by molecules of the living body. For this reason, measurement target molecules in the portion irradiated with light are locally heated and expand, thus emitting acoustic waves. The pressure of such acoustic waves is dependent on the quantity of molecules that absorb the light. Photoacoustics is a method of measuring a molecular quantity in a living body by measuring such acoustic waves. Acoustic waves are pressure waves that propagate in a living body and have a characteristic of undergoing less diffusion than electromagnetic waves, and therefore photoacoustics can be said to be suited to the measurement of a blood component in a living body.

Photoacoustic measurement makes it possible to continuously monitor the glucose concentration in blood. Furthermore, photoacoustic measurement does not require a blood sample, and does not cause the measurement subject discomfort.

### CITATION LIST

### Patent Literature

[PTL 1] Japanese Patent Application Publication No. 2010-104858.

[PTL 2] Japanese Patent Application Publication No. 2016-182160.

### SUMMARY

### Technical Problem

However, in photoacoustic measurement of glucose in a human body, acoustic characteristics may change as a result of an attachment state of a device changing due to body movement, for example. If acoustic characteristics change as described above, there is a problem in that a non-continuous point (singular point) occurs in a measurement result obtained in time series and changes in the concentration cannot be accurately known (monitored).

Embodiments of the present invention can solve the foregoing problems, and an object of embodiments of the present invention is to make it possible to more accurately know changes in the concentration of a component in a human body using photoacoustics.

### Means for Solving the Problem

A component concentration measuring device according to embodiments of the present invention includes: a light emitting unit configured to emit a light beam having a wavelength that is absorbed by a measurement target substance toward a measurement site of a measurement subject; a detection unit configured to detect a photoacoustic signal generated in the measurement site irradiated with the light beam emitted from the light emitting unit, in time series; a measurement unit configured to be attached to the measurement subject and measure acceleration in time series; a body movement calculation unit configured to obtain a magnitude of body movement of the measurement subject based on acceleration measured by the measurement unit; a singular point extraction unit configured to extract, as a singular point, a time point at which a magnitude of body movement obtained by the body movement calculation unit exceeds a threshold value; and a matching unit configured to rectify a change in the photoacoustic signal between before and after a singular point extracted by the singular point extraction unit.

In an example of a configuration of the component concentration measuring device, the matching unit subtracts a change amount by which the photoacoustic signal has changed before and after the singular point extracted by the singular point extraction unit from the photoacoustic signal after the singular point to rectify the change in the photoacoustic signal between before and after the singular point.

In an example of a configuration of the component concentration measuring device, the component concentration measuring device further includes: a determination unit configured to determine whether or not a change amount by which the photoacoustic signal has changed before and after a singular point extracted by the singular point extraction unit exceeds a threshold value; and a selection unit configured to select a singular point for which it is determined by the determination unit that the change amount exceeds the threshold value, from singular points extracted by the singular point extraction unit, wherein the matching unit rectifies a change in the photoacoustic signal between before and after the singular point selected by the selection unit.

In an example of a configuration of the component concentration measuring device, the component concentration measuring device further includes a threshold value control unit configured to increase the threshold value used in the singular point extraction unit if it is determined by the determination unit that the change amount does not exceed the threshold value, with respect to a singular point.

In an example of a configuration of the component concentration measuring device, the component concentration measuring device further includes a concentration calculation unit configured to obtain a concentration of the substance with use of the photoacoustic signal.

In an example of a configuration of the component concentration measuring device, the substance is glucose, and the light emitting unit emits the light beam having a wavelength absorbed by glucose.

### Effects of Embodiments of the Invention

As described above, according to embodiments of the present invention, a change in a photoacoustic signal between before and after a singular point that is determined based on the magnitude of body movement of a measurement subject is rectified, and therefore it is possible to more accurately know changes in the concentration of a component in a human body using photoacoustics.

### BRIEF DESCRIPTION OF THE DRAWINGS

FIG. 1 is a configuration diagram showing a configuration of a component concentration measuring device according to a first embodiment of the present invention.

FIG. 2 is a configuration diagram showing a more detailed configuration of the component concentration measuring device according to the first embodiment of the present invention.

FIG. 3 is a configuration diagram showing a configuration of a component concentration measuring device according to a second embodiment of the present invention.

FIG. 4A is a characteristic diagram showing a time series of change in a composite acceleration obtained by a body movement calculation unit based on acceleration measured by a measurement unit.

FIG. 4B is a diagram showing an example of operations of a determination unit and a selection unit.

FIG. 4C is a diagram showing an example of an operation of a matching unit.

### DETAILED DESCRIPTION OF ILLUSTRATIVE EMBODIMENTS

The following describes a component concentration measuring device according to embodiments of the present invention.

### First Embodiment

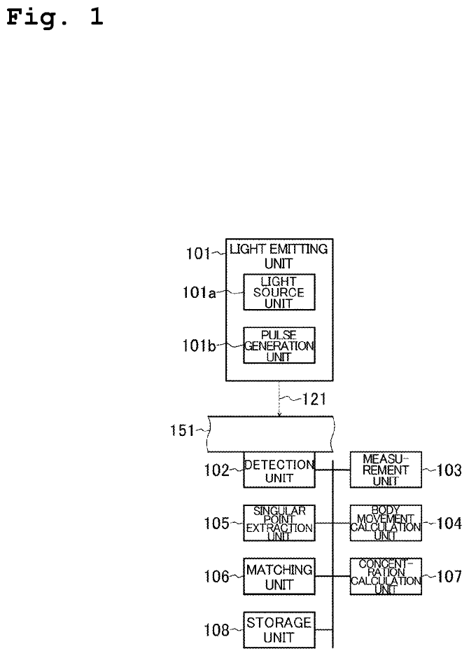

First, a component concentration measuring device according to a first embodiment of the present invention will be described with reference to FIG. 1. This component concentration measuring device includes a light emitting unit 101, a detection unit 102, a measurement unit 103, a body movement calculation unit 104, a singular point extraction unit 105, a matching unit 106, a concentration calculation unit 107, and a storage unit 108.

The light emitting unit 101 generates a light beam 121 having a wavelength that is absorbed by the measurement target substance, and emits the generated light beam 121 toward a measurement site 151 of a measurement subject. For example, in the case where the measurement target substance is glucose in blood, the light emitting unit 101 includes a light source unit 101a that generates the light beam 121 having a wavelength that is absorbed by glucose, and a pulse generation unit 101b that converts the light beam 121 generated by the light source into pulsed light that has a pre-set pulse width.

Note that glucose exhibits a property of absorbing light in wavelength bands near 1.6 μm and 2.1 μm (see PTL 1). If glucose is the measurement target substance, the light beam 121 emitted by the light emitting unit 101 is a light beam having a pulse width of 0.02 seconds or longer.

The detection unit 102 detects a photoacoustic signal generated in the measurement site that was irradiated with the light beam 121, in time series. The detection unit 102 can be a unit that employs a piezoelectric effect or an electrostrictive effect (e.g., a crystal microphone, a ceramic microphone, or a ceramic ultrasonic sensor), a unit that employs electromagnetic induction (e.g., a dynamic microphone or a ribbon microphone), a unit that employs an electrostatic effect (e.g., a condenser microphone), or a unit that employs magnetostriction (e.g., a magnetostrictive vibrator). For example, in the case of employing a piezoelectric effect, the unit includes a crystal made of a frequency flat-type electrostrictive element (ZT) or PVDF (polyvinylidene fluoride). The detection unit 102 can be constituted by a PZT that includes an FET (Field Effect Transistor) amplifier. The photoacoustic signal detected in time series by the detection unit 102 is stored in the storage unit 108 along with information indicating the time when the photoacoustic signal was measured.

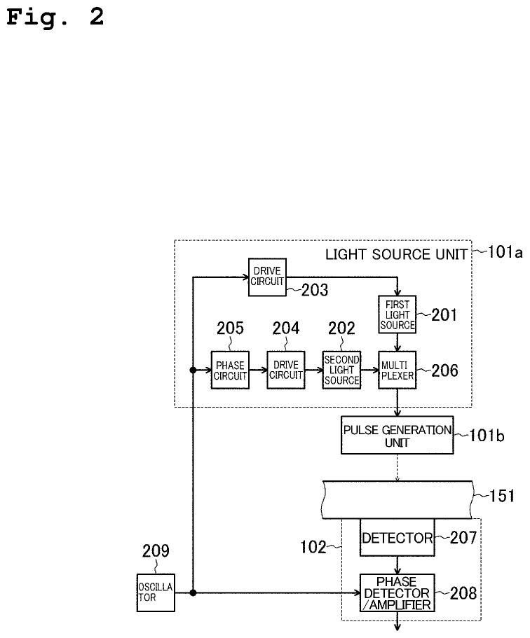

The following is a more detailed description of the light emitting unit 101 and the detection unit 102 with reference to FIG. 2. First, the light source unit 101a includes a first light source 201, a second light source 202, a drive circuit 203, a drive circuit 204, a phase circuit 205, and a multiplexer 206. Also, the detection unit 102 includes a detector 207, a phase detector/amplifier 208, and an oscillator 209.

The oscillator 209 is connected to the drive circuit 203, the phase circuit 205, and the phase detector/amplifier 208 by signal lines. The oscillator 209 transmits signals to the drive circuit 203, the phase circuit 205, and the phase detector/amplifier 208.

The drive circuit 203 receives the signal transmitted by the oscillator 209 and supplies drive power to the first light source 201 so as to cause the first light source 201 to emit light whose intensity has been modulated in synchronization with the frequency of the received signal. The first light source 201 is a semiconductor laser, for example.

The phase circuit 205 receives the signal transmitted by the oscillator 209, applies a 180-degree phase change to the received signal, and transmits the resulting signal to the drive circuit 204 via a signal line.

The drive circuit 204 receives the signal transmitted by the phase circuit 205 and supplies drive power to the second light source 202 so as to cause the second light source 202 to emit light whose intensity has been modulated in synchronization with the frequency of the received signal and the 180-degree phase changed signal received from the phase circuit 205. The second light source 202 is a semiconductor laser, for example.

The first light source 201 and the second light source 202 output light beams that have mutually different wavelengths, and the output light beams are each guided to the multiplexer 206 by an optical wave transmitting means. The wavelengths of the first light source 201 and the second light source 202 are set such that the wavelength of one of the light beams is a wavelength absorbed by glucose and the wavelength of the other light beam is a wavelength absorbed by water. Also, the wavelengths are set so as to have equivalent extents of absorption.

The light beam output by the first light source 201 and the light beam output by the second light source 202 are multiplexed into one light beam in the multiplexer 206, and the one light beam is then incident on the pulse generation unit 101b. The pulse generation unit 101b can be constituted by a light chopper, for example. Upon receiving the light beam, the pulse generation unit 101b emits the incident light beam toward the measurement site 151 as pulsed light that has a predetermined pulse width.

The detector 207 detects the photoacoustic signal generated at the measurement site 151, converts the photoacoustic signal into an electrical signal, and transmits the electrical signal to the phase detector/amplifier 208 via a signal line. The phase detector/amplifier 208 receives a synchronization signal necessary for synchronous detection from the oscillator 209, receives the electrical signal that is proportional to the photoacoustic signal from the detector 207, performs synchronous detection, amplification, and filtering, and outputs an electrical signal that is proportional to the photoacoustic signal. The electrical signal (photoacoustic signal) measured in time series and processed as described above is stored in the storage unit 108 along with information indicating the time when the electrical signal was measured.

The intensity of the signal output by the phase detector/amplifier 208 is proportional to the quantities of light absorbed by components (glucose and water) at the measurement site 151 when irradiated with the light beams output by the first light source 201 and the second light source 202, and therefore the intensity of the signal is proportional to the quantities of such components at the measurement site 151. The concentration calculation unit 107 therefore obtains the quantity (concentration) of a measurement-target substance (glucose) component in the blood at the measurement site 151 based on a measured value of the intensity of the output signal (photoacoustic signal).

As described above, two beams of light that have been subjected to intensity modulation using signals that have the same frequency are used in order to eliminate the influence of the non-uniformity of frequency characteristics when using multiple light beams, which is a problem that occurs in the case where intensity modulation is performed using signals that have different frequencies.

However, non-linear absorption coefficient dependence of photoacoustic signals, which is a problem in measurement using photoacoustics, can be resolved by performing measurement using light beams that have different wavelengths but have the same absorption coefficient as described above (see PTL 1).

Next, the measurement unit 103 is attached to the measurement subject to measure acceleration in time series. The measurement unit 103 is constituted by a well-known acceleration sensor and is attached to the measurement subject to measure acceleration. The measurement unit 103 obtains a time series of acceleration by periodically measuring acceleration in three directions of mutually orthogonal X, Y, and Z axes at a sampling rate of 25 Hz, for example.

The body movement calculation unit 104 obtains the magnitude of body movement that indicates the magnitude of a motion of the measurement subject based on acceleration measured by the measurement unit 103. For example, a composite acceleration that is obtained by combining gravitational accelerations X, Y, and Z [m/s²] in the three axes (x, y, and z axes) measured by the measurement unit 103 is taken to be the magnitude of body movement. It is known that a composite acceleration at the time of rest is about 9.8 [m/s²]. The obtained magnitude of body movement is stored in the storage unit 108 along with time information.

The body movement calculation unit 104 can also use a variance value of time series data of acceleration measured by the measurement unit 103, as an indicator of the magnitude of body movement, referring to a technology described in PTL 2. Assume that i represents a positive integer that increases by one every time acceleration data is sampled from a measurement start time point (i=1, 2, . . . ). For example, the variance value is expressed as follows where aᵢ represents the value of an acceleration norm obtained by the measurement unit 103 at an i-th measurement time point tᵢ, the population is 50 pieces of time series data of acceleration, Aᵢ represents an average, and Sᵢ² represents the variance value.

$${A}_{i} = \frac{( {a}_{i - 24} + … + {a}_{i - 1} + {a}_{i} + {a}_{i + 1} + … + {a}_{i + 25} )}{50} \\ {S}_{i}^{2} = \frac{1}{50} {\sum }_{k = - 24}^{25} {( {a}_{i + k} - {A}_{i} )}^{2} \qquad Equation 1$$

The singular point extraction unit 105 extracts, as a singular point, a time point at which the magnitude of body movement obtained by the body movement calculation unit 104 exceeds a threshold value. For example, the singular point extraction unit 105 extracts, as a singular point, a time point at which a composite acceleration obtained by the body movement calculation unit 104 deviates from a pre-set threshold value. The extracted singular point is stored in the storage unit 108, for example.

The matching unit 106 rectifies a change in the photoacoustic signal between before and after the singular point extracted by the singular point extraction unit 105. The matching unit 106 subtracts a change amount by which the photoacoustic signal has changed before and after the extracted singular point from the photoacoustic signal after the singular point to rectify the change in the photoacoustic signal between before and after the singular point. Thus, a non-continuous point can be suppressed in a measurement result obtained in time series.

According to the above-described first embodiment, even if acoustic characteristics change as a result of an attachment state of the device changing due to body movement, it is possible to eliminate a non-continuous point from a measurement result obtained in time series. As a result, it is possible to more accurately know changes in the concentration of a component in a human body using photoacoustics.

### Second Embodiment

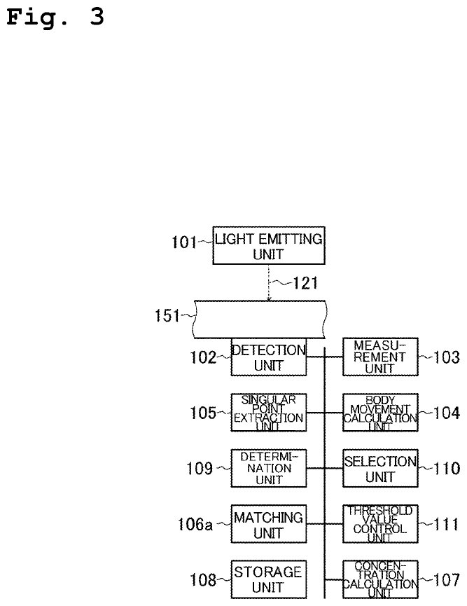

Next, a component concentration measuring device according to a second embodiment of the present invention will be described with reference to FIG. 3. This component concentration measuring device includes the light emitting unit 101, the detection unit 102, the measurement unit 103, the body movement calculation unit 104, the singular point extraction unit 105, a matching unit 106a, the concentration calculation unit 107, the storage unit 108, a determination unit 109, a selection unit 110, and a threshold value control unit in.

The light emitting unit 101, the detection unit 102, the measurement unit 103, the body movement calculation unit 104, and the concentration calculation unit 107 are similar to those in the above-described first embodiment and descriptions of which are omitted.

The determination unit 109 determines whether or not a change amount by which the photoacoustic signal has changed before and after a singular point extracted by the singular point extraction unit 105 exceeds a threshold value. The selection unit 110 selects a singular point for which it is determined by the determination unit 109 that the change amount exceeds the threshold value, from singular points extracted by the singular point extraction unit 105. In this case, the matching unit 106a rectifies a change in the photoacoustic signal between before and after the singular point selected by the selection unit 111.

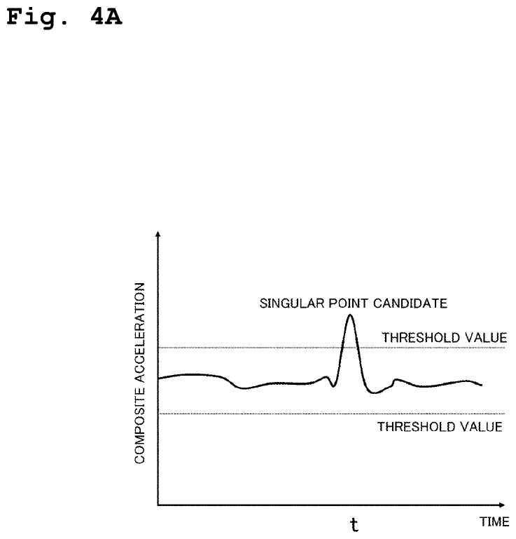

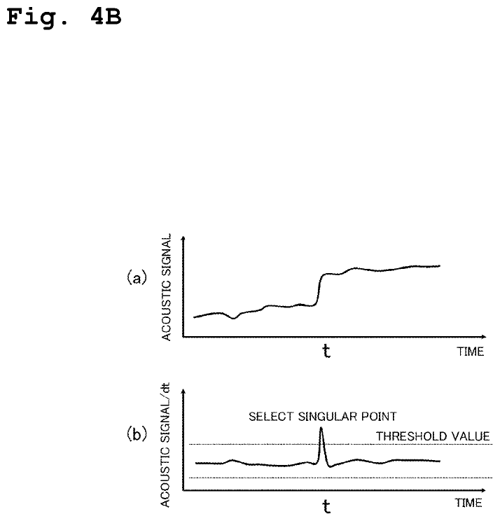

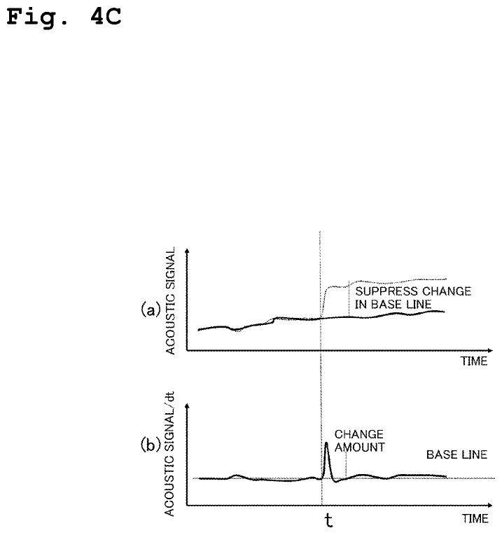

For example, in the case where a composite acceleration (body movement) obtained by the body movement calculation unit 104 based on acceleration measured by the measurement unit 103 changes with time as shown in FIG. 4A, the singular point extraction unit 105 extracts, as a singular point, a time point t at which the composite acceleration exceeds a threshold value.

Next, the determination unit 109 first obtains change amounts (derivative values) of the photoacoustic signal measured as shown in chart (a) of FIG. 4B. In this case, derivative values are obtained as shown in chart (b) of FIG. 4B. Also, the determination unit 109 determines whether or not the obtained value of the change amount at the time point t exceeds the threshold value. In this example, the derivative value at the time point t exceeds the threshold value as shown in chart (b) of FIG. 4B, and therefore the selection unit 110 selects this time point as a singular point.

When a singular point is selected as described above, the matching unit 106a subtracts the change amount obtained by the determination unit 109 from the photoacoustic signal after the singular point as shown in FIG. 4C to rectify a change in the photoacoustic signal between before and after the singular point.

Incidentally, if a singular point extracted by the singular point extraction unit 105 is not selected by the selection unit 110, a magnitude of body movement that is used as the threshold value in the singular point extraction unit 105 can be considered as being not a value that changes acoustic characteristics. Therefore, if it is determined by the determination unit 109 that the change amount does not exceed the threshold value, with respect to a singular point, the threshold value control unit 111 increases the threshold value used in the singular point extraction unit 105. As a result, it is possible to more accurately determine whether or not acoustic characteristics have changed based on the threshold value used in the singular point extraction unit 105.

As described above, according to embodiments of the present invention, a change in the photoacoustic signal between before and after a singular point that is extracted based on the magnitude of body movement of the measurement subject is rectified, and therefore it is possible to more accurately know changes in the concentration of a component in a human body using photoacoustics.

Note that the present invention is not limited to the embodiments described above, and it is clear that numerous modifications and combinations can be carried out by a person having ordinary knowledge in the art within the technical idea of the present invention.

### REFERENCE SIGNS LIST

101 Light emitting unit
101a Light source unit
101b Pulse generation unit
102 Detection unit
103 Measurement unit
104 Body movement calculation unit
105 Singular point extraction unit
106 Matching unit
107 Concentration calculation unit
108 Storage unit

1.-6. (canceled)

7. A component concentration measuring device comprising:
a light emitting device configured to emit a light beam toward a measurement site of a measurement subject, wherein the light beam has a wavelength that is absorbed by a measurement target substance;
a detector configured to detect a photoacoustic signal generated in the measurement site irradiated with the light beam in time series;
a measurement device configured to be attached to the measurement subject and measure an acceleration in time series;
a body movement calculator configured to obtain a magnitude of body movement of the measurement subject based on the acceleration measured by the measurement device;
a singular point extraction circuit configured to extract, as a singular point, a time point at which the magnitude of body movement obtained by the body movement calculator exceeds a first threshold value; and
a matching circuit configured to rectify a change in the photoacoustic signal between a time before and a time after the singular point extracted by the singular point extraction circuit.

8. The component concentration measuring device according to claim 7, wherein the matching circuit is configured to subtract an amount of the change in the photoacoustic signal between the time before and the time after the singular point extracted by the singular point extraction circuit from the photoacoustic signal after the singular point to rectify the change in the photoacoustic signal between the time before and the time after the singular point.

9. The component concentration measuring device according to claim 7, further comprising:
a determination circuit configured to determine whether or not an amount of the change in the photoacoustic signal between the time before and the time after the singular point extracted by the singular point extraction circuit exceeds a second threshold value; and
a selection circuit configured to select the singular point determined by the determination circuit to have the amount of the change in the photoacoustic signal between the time before and the time after the singular point exceeds the second threshold value from a plurality of singular points extracted by the singular point extraction circuit,
wherein the matching circuit is configured to rectify the change in the photoacoustic signal between the time before and the time after the singular point selected by the selection circuit.

10. The component concentration measuring device according to claim 9, further comprising a threshold value controller configured to increase the first threshold value used in the singular point extraction circuit when the determination circuit determines that the amount of the change in the photoacoustic signal between the time before and the time after the singular point extracted by the singular point extraction circuit does not exceed the second threshold value.

11. The component concentration measuring device according to claim 7, further comprising a concentration calculator configured to obtain a concentration of the measurement target substance using the photoacoustic signal.

12. The component concentration measuring device according to claim 7, wherein:
the measurement target substance is glucose; and
the light emitting device is configured to emit the light beam having the wavelength that is absorbed by the glucose.

13. A component concentration measuring device comprising:
a light emitting device configured to emit a light beam toward a measurement site of a person, wherein the light beam has a wavelength that is absorbed by glucose;
a detector configured to detect a photoacoustic signal generated in the measurement site irradiated with the light beam in time series;
a measurement device configured to be attached to the person and measure an acceleration in time series;
a body movement calculator configured to obtain a magnitude of body movement of the person based on the acceleration measured by the measurement device;
a singular point extraction circuit configured to extract, as a singular point, a time point at which the magnitude of body movement obtained by the body movement calculator exceeds a threshold value; and
a matching circuit configured to rectify a change in the photoacoustic signal between a time before and a time after the singular point extracted by the singular point extraction circuit.

14. The component concentration measuring device according to claim 13, wherein the matching circuit is configured to subtract an amount of the change in the photoacoustic signal between the time before and the time after the singular point extracted by the singular point extraction circuit from the photoacoustic signal after the singular point to rectify the change in the photoacoustic signal between the time before and the time after the singular point.

15. The component concentration measuring device according to claim 13, further comprising a concentration calculator configured to obtain a concentration of the glucose using the photoacoustic signal.

16. A method of measuring a component concentration, the method comprising:
emitting a light beam toward a measurement site of a measurement subject, wherein the light beam has a wavelength that is absorbed by a measurement target substance;
detecting a photoacoustic signal generated in the measurement site irradiated with the light beam in time series;
measuring an acceleration in time series using a measurement device attached to the measurement subject;
obtaining a magnitude of body movement of the measurement subject based on the acceleration measured by the measurement device;
extracting, as a singular point, a time point at which the magnitude of body movement exceeds a first threshold value; and
rectifying a change in the photoacoustic signal between a time before and a time after the singular point.

17. The method according to claim 16, wherein rectifying the change in the photoacoustic signal between the time before and the time after the singular point comprises subtracting an amount of the change in the photoacoustic signal between the time before and the time after the singular point from the photoacoustic signal after the singular point.

18. The method according to claim 16, further comprising:
determining whether or not an amount of the change in the photoacoustic signal between the time before and the time after the singular point exceeds a second threshold value; and
selecting the singular point determined to have the amount of the change in the photoacoustic signal between the time before and the time after the singular point that exceeds the second threshold value from a plurality of singular points.

19. The method according to claim 18, further comprising:
determining the amount of the change in the photoacoustic signal between the time before and the time after the singular point does not exceed the second threshold value; and
increasing the first threshold value upon determining that the amount of the change in the photoacoustic signal between the time before and the time after the singular point does not exceed the second threshold value.

20. The method according to claim 16, further comprising obtaining a concentration of the measurement target substance using the photoacoustic signal.

21. The method according to claim 20, wherein the measurement target substance is glucose.
